(19) United States
(12) Patent Application Publication
(10) Pub. No.: US 2009/0137492 A1
(43) Pub. Date: May 28, 2009

(54) METHOD TO REDUCE THE RISK AND/OR SEVERITY OF ANTHRAX INFECTION

(75) Inventors: Hal Siegel, Paradise Valley, AZ (US); John Kalns, San Antonio, TX (US)

Correspondence Address: JONES DAY, 222 EAST 41ST ST, NEW YORK, NY, 10017, US

(73) Assignee: ImmuneRegen Biosciences, Inc., Scottsdale, AZ (US)

(21) Appl. No.: 12/282,471

(22) PCT Filed: Mar. 6, 2007
(86) PCT No.: PCT/US07/05668

§ 371 Date: Jan. 16, 2009

Related U.S. Application Data
(60) Provisional application No. 60/780,825, filed on Mar. 10, 2006.
(60) Provisional application No. 60/781,367, filed on Mar. 13, 2006.
(60) Provisional application No. 60/828,723, filed on Oct. 9, 2006.
(60) Provisional application No. 60/899,035, filed on Feb. 2, 2007.

(51) Int. Cl. A61K 38/08

(52) U.S. Cl. 514/15

(57) ABSTRACT

Neuropeptides are used to treat mammals who have been exposed to or who are suspected of having been exposed to spores of Bacillus anthracis. The treatment provides a dose-dependent protection from the lethal consequences of infections.

### FIELD OF THE INVENTION

The invention relates to the field of bacterial infections. In particular, it relates to spore forming bacteria. More particularly it relates to treatment of individuals who have been or who may be exposed to anthrax.

### BACKGROUND OF THE INVENTION

The disease called anthrax is caused by Bacillus anthracis, a spore-forming bacterium. Spores are protected, nonmetabolizing forms of bacteria that remain viable even under inhospitable conditions. Spores resist extremes of heat, cold, pH, and desiccation, as well as exposure to chemicals and disinfectants. Under hospitable conditions, the spores are activated (germinate) to vegetative forms and begin to reproduce. The vegetative forms elaborate one or more toxins, which generally cause the bulk of the damage to hosts. One of the toxins functions as an adenylate cyclase and another toxin functions as a zinc metalloprotease.

The disease anthrax can affect the skin (cutaneous), the lungs (inhalation), and the digestive tract (gastrointestinal). Inhalational anthrax is more lethal (50%) than gastrointestinal anthrax (25-50%) which is more lethal than cutaneous anthrax (20%). The disease can be contracted by handling or eating infected animal products, including wool and undercooked meat. Anthrax spores have also been used as a weapon by intentionally distributing spores to people.

Cutaneous anthrax presents as a small sore and then develops into a blister and then into a skin ulcer with a black area in the center. Gastrointestinal anthrax causes nausea, loss of appetite, bloody diarrhea, and fever, followed by bad stomach pain. Inhalational anthrax presents like a cold or the flu and can include a sore throat, mild fever and muscle aches. Later symptoms include cough, chest discomfort, shortness of breath, tiredness and muscle aches.

Spores, per se, do not cause disease. The spores must find a hospitable location within the host's body to geminate. Because of this biological process, symptoms typically do not develop for one to six weeks after exposure. Thus, if exposure is known of suspected, there is a window of time to take measures which might inhibit the process of germination or reproduction, thereby averting or minimizing the disease.

Because of the spore-forming nature of Bacillus anthracis, and because spores are generally refractory to the effects of antibiotics, courses of treatment for presumed exposure are typically quite long. Moreover, although there is a vaccine, it is not generally available to the public.

There is thus a continuing need in the art for tools to help reduce the risk of disease symptoms and their severity after exposure to anthrax spores.

### BRIEF SUMMARY OF THE INVENTION

According to the present invention a method is provided for reducing risk or severity of anthrax in a mammal who has been or may be exposed to anthrax spores. An effective amount of Substance P or an analog thereof is administered to a mammal that has been or may be exposed to anthrax spores. The analog is selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹,Met (0₂)¹¹]-substance P, and analogs having the amino acid backbone RPKPQQFFGLM-NH₂ (SEQ ID NO: 1). The risk or severity of anthrax symptoms is reduced.

According to another embodiment of the invention a method is provided for reducing risk or severity of anthrax in a human exposed to anthrax spores. An effective amount of [Sar⁹,Met (0₂)¹¹]-substance P is administered to airways of the human. The risk or severity of anthrax symptoms is thereby reduced.

### DETAILED DESCRIPTION OF THE INVENTION

It is a discovery of the present invention that substance P or its bioactive analogs can reduce the risk and/or the severity of symptoms of anthrax exposure. Although applicants do not intend to be limited to any particular mechanism of action, the action of substance P or its bioactive analogs may prevent the implantation or germination of the bacterial spores. Risk of death is reduced by this therapy.

Substance P (RPKPQQFFGLM-NH₂; SEQ ID NO: 1) is synthesized as a glycine-extended precursor and converted posttranslationally to the biologically active, C-terminal amide. Substance P. or a bioactive analog thereof such as Sar⁹,Met(O₂)¹¹-Substance P can be administered to treat individuals who have been exposed or who are suspected of having been exposed to spores of B. anthracis. The bioactive analog can be selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met(O₂)¹¹-Substance P, and [p-Cl-Phe^{7,8}]-substance P. Other compounds which function in the same way can be identified by their ability to compete with substance P for binding to its receptor (NK-1) or for their ability to agonize the NK-1 receptor. Compounds which have the same amino acid backbone as substance P can be routinely modified and tested for receptor agonist activity. Routine assays for such activities are known in the art and can be used.

The substance P or analog can be administered by any method known in the art, including via aerosol inhalation. Formulations using mechanisms to ensure targeting or modifications in availability such as liposomal preparations, conjugates with targeting molecules (e.g., antibodies, lectins), and incorporation into stents, implants or other physical delivery vehicles can also be used. Intravenous, topical, intratracheal, intrabronchial, intramuscular, intranasal, subcutaneous, sublingual, and oral administrations can also be used. Suitable concentration ranges of substance P or its bioactive analog in an aerosol administered is between 1 μM and 5000 μM, including 50 μM to 500 μM, 1-10 μM, 50-100 μM, 500-1000 μM, and 1000-5000 μM. As demonstrated below, dose dependent responses were observed at 30, 100, 300 and 1000 μM. The substance P or analog can be administered alone or in combination with other agents for treating or preventing anthrax. In particular, the substance P or analog can be used to enhance the activity of other agents. Vaccines or other immunological treatments and preventatives can be enhanced by the use of the substance P or analog as an adjuvant. The adjuvant can be administered at the same time or before or after the vaccine or other immunological treatment.

The methods of the present invention can be applied to any mammal, including humans, horses, sheep, primates such as monkeys, apes, gibbons, chimpanzees, rodents such as mice, rats, guinea pigs, hamsters, ungulates such as cows. Exposure to spores can come from the ground, for example, in an agricultural setting, from wool, as in an industrial setting, such as a mill, or from eating uncooked or insufficiently cooked infected meat. A subject can be exposed to spores via inhalation, skin contact, and/or injestion, for example. Exposure can also come from weaponized spores, purposefully or accidentally distributed. Accidental exposure can come in the research or clinical laboratory setting, or in a clinical situation. Any actually or expected exposure can be an indication for treatment according to the present invention.

Additional research confirmed the findings of prophylactic efficacy of Viprovex against anthrax, and revealed another significant result. Viprovex was found to be effective at increasing survival rates in mice pretreated with anthrax. That is, Viprovex not only elicits prophylactic efficacy in an animal model, but therapeutic efficacy as well. Data revealed that treatment with Viprovex 24 hours pre-infection resulted in a 240% increase in survival rates (28.6% in untreated compared to 68.8% in treated). Treatment with Viprovex 4 hours pre-infection yielded similar results, 62.5% survival rate in treated compared to 28.6% treated. In summary, Viprovex has demonstrated efficacy of therapeutic, post-exposure treatment for anthrax exposure in a mouse model.

The scientific theory supporting these results has not been fully established at this point. Theoretically, innate immune system activation by Viprovex could be responsible for protection against anthrax infection. Mucosal membranes provide the first (innate), physical, barrier to infection and macrophage activation/phagocytosis of foreign substances presents the next component of the innate immune system. The Toll-Like Receptors (TLRs) have been shown to mediate inflammatory immune reactions and to be involved in response to anthrax and we have preliminary evidence for TLR up-regulation in response to Viprovex.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### EXAMPLES

### Example 1

Mice of the A/J strain were exposed to a single aerosolized dose of Homspera™ (Sar⁹, Met(O₂)¹¹-Substance P,) and one day later were exposed to B. anthracis Sterne spores at approximately their LD50 concentration. Homspera dosing (30, 100, 300 and 1000 uM solutions delivered via nebulizer) resulted in a dose dependent inhibition of lethality. Control animals (6) all died by 8 days, while in mice treated with 1000 uM Homspera only 2 of the 6 treated animals died over an 11 day time course and intermediate dosing presented distributed between these values, with 100 uM showing one mouse of six treated surviving, 300 uM resulting in 3 mice of 6 treated surviving.

Treatment with micromolar (uM) concentrations of Viprovex (Radilex, sar9 . . . ) either prophylactically (up to one day) or post-bacterial exposure has been demonstrated to partially protect against anthrax-induced death in an experimental murine (A/J mice) paradigm.

Animals exposed to an approximate LD70/20 dose of anthrax spores (70% of animals die at 20 days post-exposure) from four hours before, to 24 hours after initiating treatment with nebulized Viprovex showed higher survival than animals not treated. Survival ranged from approximately 20-70% of the animals that would otherwise have died. Full survival (100% of the animals that would have otherwise died) might be achievable at higher doses, as no toxicity was observed in control animals during Viprovex exposure.

While this protective mechanism is uncertain, animals treated before anthrax spore exposure were clearly protected from death as early as 5 days post-exposure, and this protection continued throughout the study (21 days). Animals treated after anthrax exposure seemed to have similar early death rates, but maintained survival from approximately day 10 onward, suggesting multiple mechanisms of Viprovex activity in protecting against anthrax inhalation.

Mechanisms such as enhanced innate immunological reactivity might account for the more aggressive impact of pretreatment with Viprovex, while an enhancement of reactive immunological mechanisms resulting from stimulation by the anthrax bacteria might be involved in later responses.

In vitro studies of Real Time PCR show elevation of cytokines, interleukins, TLR, defensins and other components of immune surveillance in response to Viprovex, and add further support to a multi-dimensional response to the potential therapeutic.

We evaluate varying pretreatment intervals before B. anthracis exposure, and evaluate peripheral blood cytokine levels and pulmonary cell function mediators as well as measures of immune system surveillance following Homspera exposure +/−B. anthracis exposure. Specifically, peripheral blood is evaluated for a change in the expression of 17 cytokines evaluated using multiplex flow cytometric analysis, i.e. via Luminex technology. Additionally, flow cytometry is used to determine changes in relative abundance of different groups of immune cells including CD4, CD8, NK and stem (CD34+) cells. Groups of 6 mice are evaluated 2, 4, 24 and 72 hours after Homspera. A control group used for comparison receives Homspera vehicle via nebulizer and is sacrificed at the same times as the treatment group. A single sham group does not receive any treatment but otherwise is handled identically. Other treatment groups receive molar equivalent doses of SP or a selected NK1 receptor antagonist to verify the Homspera actions are NK1-receptor mediated.

### Example 2

### An Immunostimulant Activity that Preferentially does not Elevate IL-6 and Implications

Viprovex™ provides therapeutic efficacy in animal models of infection by influenza virus. Studies looking at potential mechanisms have examined cellular components of the host immune system and have found that in both animals and in cultured cells, Viprovex™ causes differential activation of components of the innate immune system, supporting the whole animal findings and suggesting that Viprovex™ may be capable of thwarting the immune system over-stimulation which might underlie the severe lethality of H5N1 influenza (“bird flu”).

Reporting in Nature (Kobasa D et al, (2007) Nature 445: 319-323), scientists from Canada's Public Health Agency, in Winnipeg, Manitoba, have reported on what might be the underlying mechanism by which some influenza viruses can be so deadly. Both the 1918 flu virus (Spanish Flu), which caused a pandemic in which an estimated 40-100 million died worldwide, and the threatening avian flu virus, both seem to cause a “cytokine storm” in infected individuals. Normally, in the presence of an invading virus, the immune cells release chemicals (such as cytokines and chemokines) that cause inflammation, attract more immune cells and stimulate the development of cells and antibodies that attack the invading virus. Cytokine storm occurs when an infected individual's immune system remains activated against the virus beyond the point of being helpful to where the immune response turns deadly. Highly elevated levels of cytokines overstimulate the immune system resulting in massive pulmonary inflammation and fluid accumulation, vascular dysfunction and eventually shock and death. In cytokine storm, the body's immune system fights to rid itself of the virus, but somehow escapes from the normal controls that prevent an overzealous immune system from killing its owner.

As noted in the Nature publication, there are other disease conditions in which a hyperactive immune system is involved, and drugs under development for treating those conditions might be beneficial in treating a pandemic influenza infection that could trigger cytokine storm. Specifically mentioned as an immune response control point is the cytokine interleukin-6 (IL-6), which helps activate lymphocytes and increase antibody production. It has been identified as central to regulation of the immune system, inflammation and hematopoiesis. [Nishimoto N and Kishimoto T (2006) Nature Clinical Practice 2:11; 619-626]

While normal production and release of IL-6 may be integral to functioning immune and hematopoietic systems, overproduction has been reported to be anti-apoptotic and result in unregulated cell growth, specifically associated with the blood cancer malignant myeloma.

Cellular genes that code for immune system proteins were quantified using PCR; Viprovex™ was shown to elevate multiple components of the host immune system, among them IL-1beta, IL-6, IL-10, TNF-alpha and a variety of cellular sensors such as Toll-Like Receptors (TLRs) and intracellular pathogen recognition molecules such as nucleotide-binding oligomerization domains (NODs) and regulatory factors such as SOCS (suppressor of cytokine signaling). The interleukin mRNA elevations were noteworthy in that IL-1 and IL-10 increased 28× and 21× more than IL-6.

Viprovex ™

| ug/ml | IL-1beta | IL-10 | IL-6 |
|---|---|---|---|
| 0 | 1 | 1 | 1 |
| 5 | 1.071773 | 2 | 1.414214 |
| 12.5 | 337.794 | 256 | 6.062866 |
| 25 | 955.4258 | 724.0773 | 34.29675 |
| 125 | 84.44851 | 10.55606 | 3.732132 |

Animal studies in cotton rat confirm that in the lungs of animals treated with Viprovex™ (and more resistant to the effects of H3N2/Wuhan influenza virus), IL-6 levels do not significantly increase.

| Il-6 | avg | SD | SE |
|---|---|---|---|
| 2.178 | 2.195 | 0.152 | 0.088 |
| 2.052 | | | |
| 2.354 | | | |
| 2.458 | 2.284 | 0.462 | 0.267 |
| 2.633 | | | |
| 1.759 | | | |
| 7.483 | 3.632 | 3.336 | 1.928 |
| 1.791 | | | |
| 1.623 | | | |
| 1.253 | 1.959 | 0.629 | 0.363 |
| 2.458 | | | |
| 2.167 | | | |
| 3.013 | 1.81 | 1.596 | 0.922 |
| 0 | | | |
| 2.416 | | | |

P-Values (2 sample unequal variance, 2-tailed)

| IL-6 | |
|---|---|
| Mock | 0.78 |
| 30 | 0.534 |
| 100 | 0.593 |
| 300 | 0.718 |

In contrast, other immunostimulatory cytokines are elevated significantly.

This relative underexpression of IL-6 relative to other immunostimulatory cytokines in these macrophage-like cells and in animals treated with Viprovex™, demonstrates that Viprovex™ stimulates the immune system in a preferential way that conveys a lower tendency to trigger cytokine storm.

I claim:

1. A method for reducing risk or severity of anthrax in a mammal who has been or may be exposed to anthrax spores, comprising:
administering an effective amount of Substance P or an analog thereof to a mammal that has been or may be exposed to anthrax spores, wherein said analog is selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, [p-Cl-Phe^{7,8}]-substance P, [Sar⁹,Met (0₂)¹¹]-substance P, and analogs having the amino acid backbone RPKPQQFFGLM-NH₂, whereby the risk or severity of anthrax symptoms is reduced.

2. The method of claim 1 wherein Substance P is administered.

3. The method of claim 1 wherein [Met-OH¹¹]-Substance P is administered.

4. The method of claim 1 wherein [Met-OMe¹¹]-Substance P is administered.

5. The method of claim 1 wherein [Nle¹¹]-Substance P is administered.

6. The method of claim 1 wherein [Pro⁹]-Substance P is administered.

7. The method of claim 1 wherein [Sar⁹]-Substance P is administered.

8. The method of claim 1 wherein [Tyr⁸]-Substance P is administered.

9. The method of claim 1 wherein [p-Cl-Phe^{7,8}]-Substance P is administered.

10. The method of claim 1 wherein [Sar⁹,Met (0₂)¹¹]-Substance P is administered.

11. The method of claim 1 wherein the step of administering is performed by administration of an aerosol to the mammal's nose.

12. The method of claim 1 wherein the step of administering is performed by injection into a muscle of the mammal.

13. The method of claim 1 wherein the step of administering is performed by topical administration.

14. The method of claim 1 wherein the step of administering is performed by intravenous administration.

15. The method of claim 1 wherein the step of administering is performed by administration of an aerosol to the mammal's lungs.

16. The method of claim 1 wherein the mammal is a human.

17. The method of claim 1 wherein the mammal is a bovid.

18. The method of claim 1 wherein the mammal is a dog.

19. The method of claim 1 wherein the mammal is a cat.

20. The method of claim 1 wherein the human was exposed to anthrax spores.

21. The method of claim 1 wherein the human may be exposed to anthrax spores.

22. The method of claim 1 wherein the human was exposed to anthrax spores via skin contact.

23. The method of claim 1 wherein the human was exposed to anthrax spores by inhalation.

24. The method of claim 1 wherein the human was exposed to anthrax spores by ingestion.

25. A method for reducing risk or severity of anthrax in a human exposed to anthrax spores, comprising:
administering an effective amount of [Sar⁹,Met (O₂)¹¹]-substance P, to airways of the human, whereby the risk or severity of anthrax symptoms is reduced.

26. The method of claim 25 wherein the [Sar⁹,Met (O₂)¹¹]-substance P is delivered in an aerosol.


SEQUENCE LISTING (1 sequence)

SEQ ID NO: 1 - protein, 11 aa, Artificial Sequence
  Substance P
RPKPQQFFGLM